(19) United States
(12) Patent Application Publication
(10) Pub. No.: US 2011/0097796 A1
(43) Pub. Date: Apr. 28, 2011

(54) METHOD FOR FREEZING AND STORING CELLS

(75) Inventor: Alexander Loa, Henstedt-Ulzburg (DE)

(73) Assignee: CCS CELL CULTURE SERVICE, Hamburg (DE)

(21) Appl. No.: 12/915,963

(22) Filed: Oct. 29, 2010

Related U.S. Application Data
(62) Division of application No. 11/794,188, filed on Jun. 22, 2007.

(30) Foreign Application Priority Data
Dec. 22, 2004 (DE) 102004061947.6

(51) Int. Cl. C12N 5/07

(52) U.S. Cl. 435/374

(57) ABSTRACT

This invention relates to a method for freezing and storing cells in a container, and relates to frozen cells in freezing medium in a container.

This application is a Divisional of U.S. application Ser. No. 11/794,188, filed Jun. 22, 2007, which is a 371 U.S. National Entry of PCT/EP2005/013170, filed Dec. 8, 2005, which claims priority to German Application No.: 102004061947.6, filed Dec. 22, 2004, each of which are herein incorporated by reference in their entireties.

### FIELD OF THE INVENTION

The invention is related a method for freezing and storing of cells in a container. In addition, the invention is related to frozen cells in freezing medium in a container.

### BACKGROUND OF THE INVENTION

The freezing of cells is a method that is often used in laboratories, which work in cell biology (Parkes A. S., Proc. R. Soc. Lond. B. Biol. Sci. 147 (929): 424-426, 1957). In order to avoid the detrimental effects of freezing, which are predominantly caused by the gener'ation of ice crystals, cryoprotective substances such as glycerol or DMSO are usually added to the freezing medium (Pomerat C. M. und P. S. Moorhead, Tex. Rep. Biol. Med. 14 (2): 237-253, 1956; Bouroncle B. A., Proc. Soc. Exp. Biol. Med. 119 (4): 958-961, 1965; Murthy S. S., Cryobiology 36 (2): 84-96, 1998). The cells suspended in freezing medium are slowly frozen down with a cooling rate from approximately 1° C. per minute up to −80° C. per minute. The slow cooling down is achieved by individual isolation of the cryotubes, e. g. by a polystyrene box having a suitable wall thickness. The cells frozen in this way can only be preserved for a short period of approximately 2 to 4 months.

For a long term preservation at lowest temperatures, the transfer of the samples in liquid nitrogen (−196° C.) or in its gas phase (−150° C. to −160° C.) needs to be done, where in theory an unlimited storage life of the frozen cells is possible (Lindl T., Zell-und Gewebekultur, Spektrum Akademischer Ver'lag GmbH, 4: 110-113, 2000). Animal and human cells are used in cell-based assays in “high-throughput-screening” (HTS). A continuous culture of the cells according to techniques known in the art is required for this purpose, so that sufficient numbers of cells of constant quality are provided regularly, which can be seeded in the containers used in the assays, usually multiwell plates. However, up to now it is not possible to prepare the cells in the containers, to store them in this form at −80° C., for long terms and to keep them in this way in any amount for the assay. The storage life of cells, which have been frozen at −80° C., is limited to 2 to 4 months. In addition, the storage of multiwell plates and other containers in liquid nitrogen is technically difficult and only possible with a big requirement for space. Moreover, the need to freeze the cells down slowly, as described above, makes the processing of a high number of containers more difficult.

### BRIEF SUMMARY OF THE INVENTION

The problem underlying the invention is to provide a method, which allows for the provision of cells for use in cell-based assays in the respective containers in sufficient numbers and constant quality and for long term storage.

The invention solves this problem by providing a method according to claim 1 comprising the following steps:

a) freezing the cells down to a storage temperature,

b) storing at said storage temperature,

wherein the storing is performed under gas tight sealing and/or under reduced oxygen partial pressure as compared to atmospheric pressure.

### BRIEF DESCRIPTION OF THE DRAWINGS

FIG. 1 shows the result of the experiment of Example 1.

FIG. 2 shows graphically the result of Example 2.

FIG. 3 shows the raw data of Example 2.

FIG. 4 shows the result of Example 3.

### DEFINITIONS

First of all, a number of terms are explained, which are used in the invention.

The term “cell” according to the invention can be a eukaryotic cell that means a cell with a true cell nucleus, as well as a number of characteristic differentiations of the cytoplasm. Such a eukaryotic cell according to the invention can be of animal or human origin.

The term “container” used according to the invention can be a “well” (cavity) of a multiwell plate, as well as the entirety of the plate. Likewise, the term “container” can also refer to other containers used in cell culture, such as e.g. cell culture dishes or cell cultures flasks of a number of sizes, which are suitable for culturing cells.

The term “storage temperature” according to the invention refers to that temperature, at which the cells are stored, preferably in a deep freezer. The cells can be stored at this storage temperature for at least one month up to more 30 than 12 months.

The term “gas tight sealing” according to the invention refers to the sealing of the container, in which the cells are frozen, which is capable of keeping up an oxygen partial pressure that is reduced compared to atmospheric pressure, or of keeping protective gas in the container.

The term “atmospheric pressure” according to the invention refers to the total air pressure of the surrounding atmosphere consisting of air, which is 1 bar.

The term “oxygen partial pressure” according to the invention refers to the partial pressure of oxygen in gas form of a total pressure of an atmosphere consisting of air. The oxygen partial pressure in an atmosphere consisting of air, which has a pressure of 1 bar, is 0.21 bar. The term “multiwell plate” according to the invention refers to a plate of a suitable material, such as polystyrene, which comprises a number of “wells” (cavities) for taking up the cells in cell culture medium. Such a plate can have 6, 12, 24, 48, 96, 384 or 1 536 of such wells.

The term “thermal conductivity” according to the invention has to be understood by its known physical definition. A measure for the thermal conductivity of a compound is its thermal conductivity value. Metals have a high thermal conductivity compared to e. g. glass. The thermal conductance always proceeds from regions of high temperature to regions of low temperature.

The term “high-throughput-screening” (HTS) refers to assaying a plurality of compounds, cells or suchlike in a relatively short period of time. For example, the effect of compounds, cells or suchlike on cells in the wells of suitable containers, preferably of a multiwell plate, can be tested in a cell-based assay. Preferably, this can be performed with a plurality of compounds or cells or suchlike with a high throughput rate.

The term “freezing medium” comprises solutions, which are suitable for protecting living cells from cryo-damage during freezing. Such solutions can comprise culture medium and cryoprotective substances (“cryprotectives”), which can be solvents such as dimethyl sulfoxide (DMSO) or glycerol.

The term “cell-based assay” according to the invention is used for an experiment (“assay”), in which living cells can be used. The cells can be used in this assay directly after thawing in the wells of a suitable container. Thus, the cell-based assay can be performed directly in the wells of this container.

The term “vitality” according to the invent.ion refers to the fraction of living cells in a container to be tested, e.g. of a multiwell plate, in comparison to cells in a nonfrozen container, the “control container”, such as a multiwall plate, as reference. The determinat.ion of the fraction of living cells can either be made by staining the cell nucleus (dye exclusion test, Thumm W., Z. Krebsforsch. 60 (1): 25 91-93, 1954), or by testing for metabolic activity (Glass R. H., S. A. Ericsson et al., Fertil. Steril. 56 (4): 743-746, 1991).

### DETAILED DESCRIPTION OF THE INVENTION

The invention has shown that. a method as outlined above is very suitable for providing cells for use in cell-based assays in suitable containers in sufficient numbers and constant quality and for long term storage.

In the following the invention is described further.

Subject of the invention is a method for freezing and storing cells in a container comprising the steps of:

a) freezing the cells down to a storage temperature,

b) storing at said storage temperature, characterized in that the storage is performed under gas tight sealing and/or under reduced oxygen partial pressure as compared to atmospheric pressure.

Preferably, cells of a cell culture that is in exponential growth phase are used for freezing the cells down. Prior to harvesting, preferably such an amount of cells is in the container that evenly covers the floor of the container. In the case of a well of a 96-well plate this preferably equates to 80 to 200 μl, more preferably 85 to 1.90 μl, most preferably 93 to 188 μl of culture medium with cells per 20 cm² surface.

After harvesting the cells can be re-suspended in freezing medium at a cell density of preferably 0.05−10×10⁶ cells, more preferably at 0.1−5×10⁶ cells per ml. The freezing medium can comprise culture medium, as well as a cryoprotective. The cryoprotective can be glycerol or dimethyl sulfoxide (DMSO). The final concentration of the cryoprotective can be 1 to 20% per vol., preferably 2 to 15% per vol., most preferably 4 to 10% per vol. Examples for suitable freezing media are a mixture of 95% DMEM (Dulbecco's Modified Eagels Medium) complete medium, 5% glycerol, as well as of 95% DMEM complete medium, 5% DMSO, wherein complete medium can consists of DMEM, 4 mM LGlutamine, 2 mM sodium pyruvate and 10% fetal calf serum (FeS).

The culture medium used in the freezing medium can be a different medium than DMEM. Every other medium used in cell cult.ure can be used, such as RPMI or neurobasal medium. A salt solution in place of the cell culture medium can also be used in the freezing medium, such as HBSS (Hank's Balanced Salt Solution) or other common salt solutions.

The cells resuspended in freezing medium can at the most be frozen 60 min, preferably at the most 45 min, most. Preferably at the most 30 min after resuspending.

The reduction of the oxygen partial pressure can be performed by reducing the pressure and/or by gassing with a protective gas.

The reduction of the oxygen partial pressure can preferably be done prior to freezing.

The oxygen partial pressure in the container can be reduced to 52.5 mbar to 0.21 mbar, preferably to 26 mbar to 0.21 mbar, most preferably to 13 mbar to 0.21 mbar.

The gas tight sealing of the container according to the invention can be made by shrink-wrapping the container in a gas tight film tube or by applying a gas tight adhesive film. In doing this, a suitable container can be closed with a lid and shrink-wrapped under vacuum in a gas tight film tube that is preferably made from polyethylene. The container can be carefully moved in the tube, which is then closed at one side. Using a commercially available shrink wrapping device, the remaining air oxygen can be removed from the film tube and the film can be shrink-wrapped. Such a shrink-wrapping device can be, e.g. a Folio vacuumshrink-wrapping device. For comparison, suitable containers can be frozen under the same conditions, however under an oxygen partial pressure that is reduced compared to the atmospheric pressure.

After gas tight sealing of the container a residual volume of air referring to atmospheric pressure can remain in the container, that is preferably up to 17.5 times, more preferably up to 15 times, more preferably up to 7 times, more preferably equal to the volume of the cell suspension in the container.

For example, such a container can be a multiwell plate, preferably a 96-well plate. A residual volume of air in comparison to the atmospheric pressure can remain in the 96-well plate with a volume of 25 μl cell suspension per well under gas tight sealing, which can be most preferably up to 17.5 times the volume of the cell suspension.

The protective gas according to the invention can be selected from the group consisting of nitrogen, helium and argon.

The cells, which are frozen with the method according to the invention, can be eukaryotic cells. The cells that are frozen with the method according to the invention can be of animal or human origin. For example, these cells can be murine cells, hamster cells, monkey cells or pig cells. The cells, which are frozen with this method can further be established cell lines, as well as primary or secondary cells. The cells, which are frozen with the method according to the invention, can further be adherent cells, or cells that grow in suspension. The cells, which are frozen with this method, can comprise a plurality of cell types. These cell types can be selected from the group consisting of cells of ectodermal origin, mesodermal origin, endodermal origin and germ cells. Furthermore, the cell types can be selected from the group consisting of fibroblasts, epithelial cells, haematopoietic cells and neural cells. For example, the cells can be L-292 mouse fibroblasts, HeLacells or HCT-116 cells.

As contemplated in the invention, the cells can be frozen down to the storage temperature with a cooling rate, which can be at least 20° C. per minute, preferably at least 40° C. per minute, more preferably at least 64.5° C. per minute, more preferably at least 80° C. per minute. The cooling rate according to the invention, as compared to techniques known in the art, allows for a rapid cooling, and even for a cooling below the freezing point of the cell suspension present in the container. Together with the medium it is possible to suppress the formation of crystals in the system and to keep the solution in that glass-like state, which is necessary for storing the cells at lowest temperatures without causing damage. A cooling rate according to the invention can most preferably be 64.5° C. per minute.

The storage temperature according to the invention can be not lower than −85° C., more preferably not lower than −65° C., more preferably not lower than −60° C., more preferably not lower than −50° C. According to the invention the storage temperature can be maintained in a deep freezer. As contemplated by the invention, a particularly preferred storage temperature can be not lower than −65° C.

The freezing can be achieved by contact of at least one surface of the container with a material of suitable (high) thermal conductivity, which has been pre-cooled on a suitable temperature. The material of suitable thermal conductivity can be a metal. The suitable temperature can be between −90° C. and −50° C., preferably between −85° C. and −65° C.

The storage of the cells according to the invention can be performed without the use of liquid nitrogen. Preferably, the storage of the cells is done in a deep freezer.

The container can be selected from the group consisting of multiwell plate, cell culture dish and cell culture flask.

The multiwell plate can be selected from the group consisting of 6-well, 12-well, 24-well, 48-well, 96-well, 384-well and 1 536-well plate.

The invention further relates to frozen cells in freezing medium in a container under gas tight sealing and/or under oxygen partial pressure that has been reduced in comparison to the atmospheric pressure.

The oxygen partial pressure in the container can be 52.5 mbar to 0.21 mbar, preferably 26 mbar to 0.21 mbar, most, preferably 13 mbar to 0.21 mbar.

The cells according to the invention can be used directly after thawing in freezing medium in the container in a cell-based assay without passaging. This can be performed without washing, culturing or passaging the cells to a different culture dish. Approximately 1 to 8 hours after thawing at 37° C., culture medium can be added to the wells in the container, preferably in a suitably tempered cell incubator with suitable CO2-atmosphere. In doing so, the freezing medium surrounding the cells is diluted. Preferably, this can be a 1:1 dilution. After the addition of culture medium, the cells can be used directly in the container for a cell-based assay. This can be done without performing washing, culturing or passaging of the cells to a different container. In the following cell-based assay, controls can be done with and without the solvent that has been used as cryoprotective in the respective percentage, in order to test for a potential effect of the solvent on the assay.

The cell-based assay can be performed in the form of a high-throughput-screening (HTS). The effect of e.g. compounds, cells or suchlike can be tested on cells in wells of a container, preferably a multiwell plate. Preferably, this is performed with a plurality of compounds or cells with a preferably high throughput rate. The tested compounds can preferably be pharmacologically active compounds. The tested compounds can, in addition, be preferably ligands for cellular receptors. The tested compounds can particularly preferably have an effect of the gene expression in the cells of the wells. The tested cells can have an effect on the cells in the wells. One or more compounds or cells per well can be tested.

The frozen cells can have a vitality at a storage temperature lower than or equal to −65° C. for at least one month, preferably more than 2 months, more preferably more than 4 months, most preferably more than 12 months of above 70%, preferably above 80%, most preferably of above 95% of the vitality of cells of a non-frozen control container. In the case of large containers from which the cells can easily be removed completely, the vitality of cells can be determined by the dye exclusion assay that is known in the art (Thumm W., Z. Krebsforsch. 60 (1): 91-93, 1954). For smaller containers, from which the frozen cells can not be taken reliably for determining the vitality, the vitality assay can be performed indirectly via the determination of the metabolic activity of the cells. A substrate, e.g. resazurin, is added to the cells and is reduced by metabolically active cells. The absorption spectrum or fluorescence spectrum of the substrate is changed by the reduction, which can easily be quantified (Glass R. H., S. A. Ericsson et al., Fertil. Steril. 56 (4): 743-746, 1991). After preceding grading of the measured absorption or fluorescence by using cell suspensions with a known cell count, quantifying of the vitality can be performed. The measurement of the metabolic activity of the cells can, apart from resazurin, be 20 performed with other dyes known in the state of the art, such as MTT, XTT or neutral red.

The invention is further explained in the following examples.

### Example 1

### Materials And Devices

1. L-929 murine fibroblast cell line of ATCC (American Type Culture Collection), No.: CCL-1

2. DMEM culture medium (Dulbecco's Modified Eagels Medium)

3. L-glutamine

4. sodium pyruvate

5. fetal calf serum (FCS)

6. glycerol

7. PBS (Phosphate Buffered Saline) Trypsin I EDTA (Ethylene-diamine-tetraacetic acid), 0.5 g/l and 0.25 g/l

8. Resazurin

9. 96-well plates

10. polyethylene tube film

11. Folio vacuum shrink-wrapping device

12. sterile working bench

13. CO₂ cell culture incubator

14. −80DC deep freezer

L-292 mouse fibroblasts were cultured according to the standard protocol of the ATCC in DMEM complete medium (DMEM, 4 mM L-glutamine, 2 mmM sodium pyruvate, 10% FCS) until a sufficient number of cells was provided for freezing of the cells. For harvesting, the surfaces of the containers should be approximately 80 to 90% confluently grown with cells. The culture supernatant was decanted and the layer of cells was washed once with PBS. 1 ml trypsin/EDTA were applied on the cell layer per 25 cm² surface, equally dispersed and incubated at room temperature (RT) for 5 minutes (min). The number and vitality of the detached cells was determined, which were then pelleted at 180×g for 4 min at RT. The supernatant was discarded and the cell pellet was re-suspended at a cell density of 6×10⁵ cells/ml in freezing medium (95% DMEM complete medium, 5% glycerol). 50˜1 of the cell suspension were pipette in each well of a 96-well plate, wherein special care was taken that the cell suspension was covering the surface of the wells. The 96-well plates were closed with the lid and each of them shrink-wrapped under vacuum in a film tube made from polyethylene. The plates were carefully placed in the film tube, which was then sealed at one side. using a commercially available shrink-wrapping device the remaining air was removed from the film tube as far as possible and the film was shrink-wrapped. For comparison, 96-well plates were frozen under the same conditions, however without removing the air. The shrink-wrapped multiwell plates were placed directly on the metal floors of a −80° C. deep freezer, and frozen and stored in there. The plates were not stacked, and the entire freezing process was done rapidly. The time period from adding the freezing medium to starting the freezing process was no more than 45 min in this example.

The cells could be stored in 96-well plates under such conditions for more than 1.5 years and thawed again with a vitality of more than 90%. However, when cells were not frozen under vacuum, their storage life was limited to 2 to 4 months. The determination of the vitality was done directly in the 96-well plate using the reduction of resazurin (Glass R. H., S. A. Ericsson et al., Fertil. Steril. 56 (4): 743-746, 1991).

The cells could be stored under such conditions for more than 12 months and could be thawed in the 96-well plates with a vitality of more than 90% of a none-frozen plate. In contrast, the vitality of the control plate, which had not been treated with the method was only just above 20%.

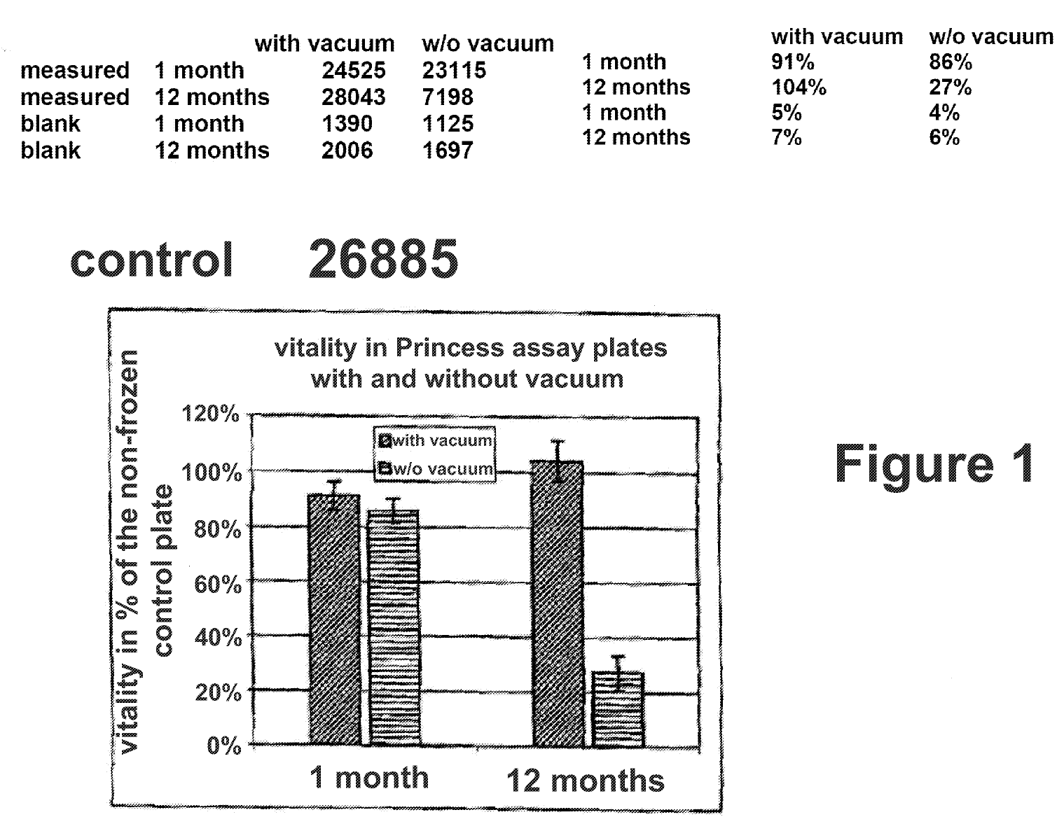

FIG. 1 shows the result of the experiment of Example 1.

### Example 2

The cell line HCT-116 was frozen using the method according to the invention. For comparison, HCT-116 cells were frozen in freezing medium with DMSO (95% DMEM complete medium, 5% DMSO) and with glycerol (95% DMEM complete medium, 5% glycerol) in 96-well plates. The frozen cells were thawed again after 12 months and the cell vitality was determined by reduction of the dye resazurin. The cell activity is given as relative signal intensity in [U].

For analyzing a HTS-assay the Z-factor is used in the state of the art. The Z-factor is a statistical value, which is calculated from the average values and the standard deviations of the measured values. An ideal assay has a Z-factor, which is around 1. Assays are preformed well between 1 and 0.5. Is the Z-factor falling below 0.5, the separating area between control and sample is getting too small, so that a clear distinction of the cell activity is not possible anymore.

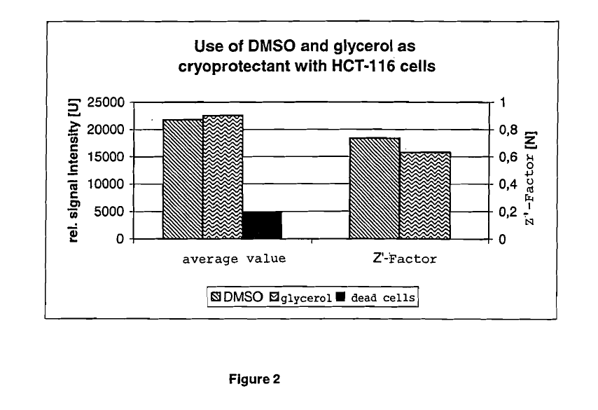

FIG. 2 shows graphically the result of Example 2.

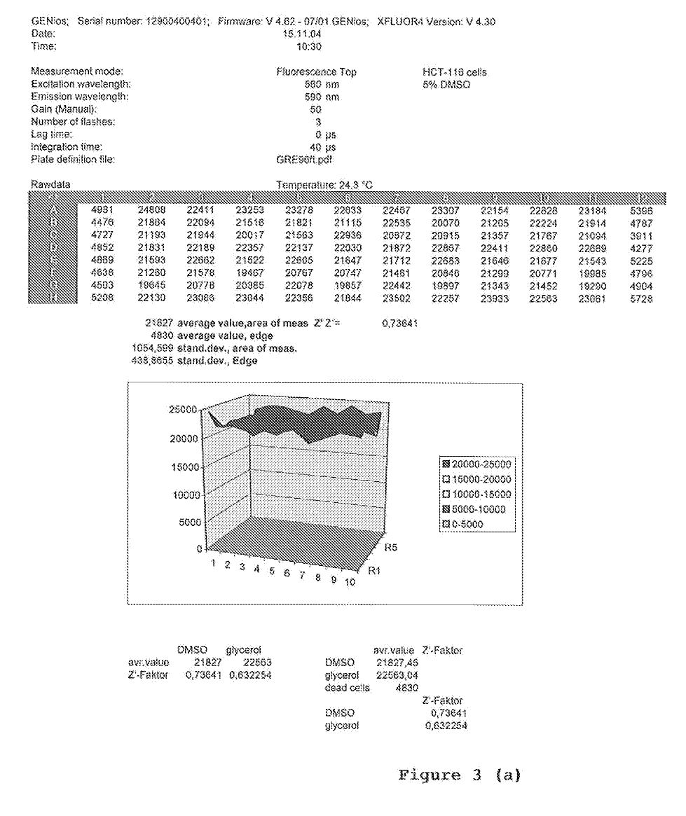

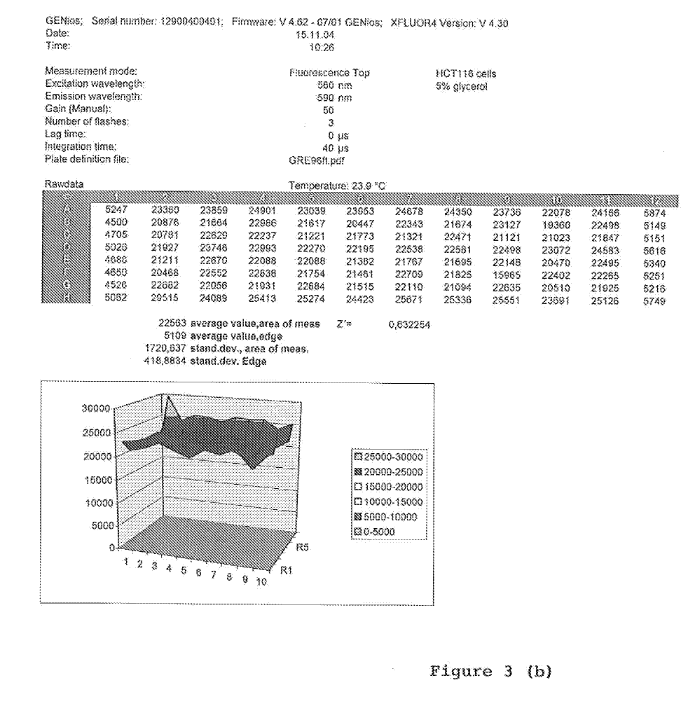

FIG. 3 shows the raw data of Example 2.

### Example 3

In order to show the functionality of cells frozen according to the invention, cells were used in a cytotoxicity as say after thawing. HeLa-cells were frozen in 96-well plates using the method according to the invention, then thawed after 13 months and their IC₅₀-value of Na₂SeO₃ determined. To do this, 12 dilutions of Na₂SeO₃ were added to the cells. The dilutions were selected such, that the concentration of Na₂SeO₃, which kills 50% of the cells, was in the middle of the tested concentration range. The depicted values are average values of a quadruplicate.

After incubating the test substance for 48 hours at cell culture conditions (37° C., 5% CO2 and high air humidity), the cell count of living cells was determined by testing for the metabolic activity of living cells. For this purpose, resazurin was used again, which is reduced in the mitochondria of metabolically active, living cells, and which turns into the fluorescent resofurin. The lC50-value is given by that concentration, at which only 50% of the cell activity can be detected.

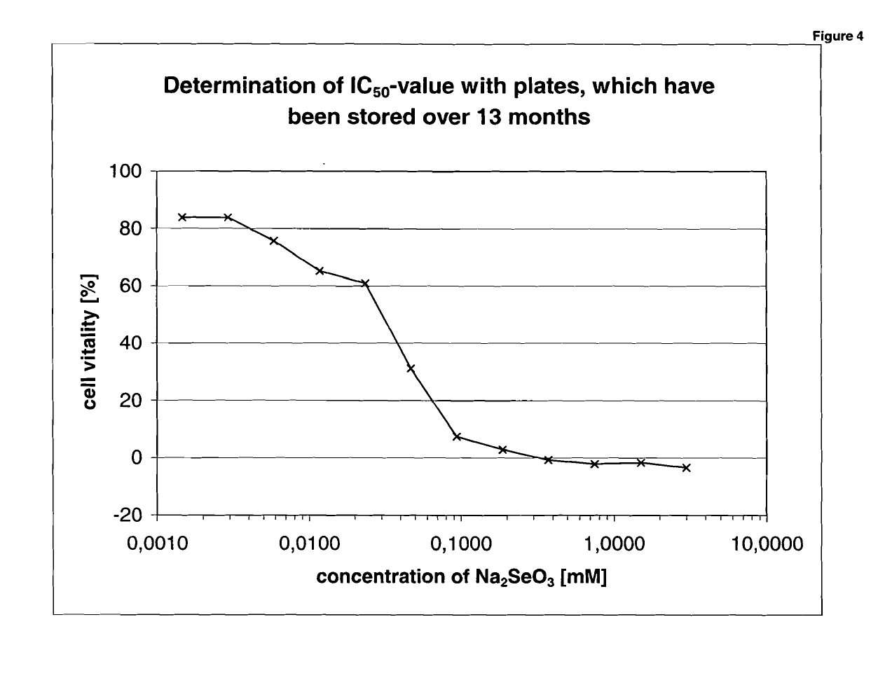

FIG. 4 shows the result of Example 3.

The examples show that the method according to the invention is particularly suitable for providing cells for use in cell-based assays in the respective containers in sufficient numbers and constant quality and for storing them for long terms.

1. A composition comprising frozen cells in freezing medium in a container under gas tight sealing and/or under an oxygen partial pressure that has been reduced in comparison to the atmospheric pressure.

2. The composition of claim 1, wherein the oxygen partial pressure in the container is between about 52.5 mbar and 0.21 mbar.

3. The composition of claim 1, wherein the oxygen partial pressure in the container is between about 26 mbar to 0.21 mbar.

4. The composition of claim 1, wherein the oxygen partial pressure in the container is between about 13 mbar to 0.21 mbar.

5. The composition of claim 1, wherein the cells can be used directly after thawing in freezing medium in the container in a cell-based assay without passaging.

6. The composition of claim 5, wherein the cell-based assay is done in the form of a high-throughput-screening (HTS).

7. The composition of claim 1, wherein after storage at a storage temperature lower than or equal to about −65° C. for at least one month, said frozen cells have a vitality of above 70% of the vitality of cells of a non-frozen control container.

8. The composition of claim 7, wherein said frozen cells have a vitality of above 80% of the vitality of cells of a non-frozen control container.

9. The composition of claim 8, wherein said frozen cells have a vitality of above 90% of the vitality of cells of a non-frozen control container.

10. The composition of claim 7, wherein said storage at said storage temperature is for at least 2 months.

11. The composition of claim 7, wherein said storage at said storage temperature is for at least 4 months.

12. The composition of claim 7, wherein said storage at said storage temperature is for at least 12 months.
